(19) United States
(12) Patent Application Publication
(10) Pub. No.: US 2009/0125331 A1
(43) Pub. Date: May 14, 2009

(54) METHODS AND SYSTEMS FOR TREATING ILLNESSES

(75) Inventors: Janne Pamsgaard, Kobenhavn O (DK); Jesper Lauridsen, Copenhagen K (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S, Valby-Copenhagen (DK)

(21) Appl. No.: 12/208,805

(22) Filed: Sep. 11, 2008

Related U.S. Application Data
(60) Provisional application No. 61/057,690, filed on May 30, 2008.
(60) Provisional application No. 60/971,975, filed on Sep. 13, 2007.

(51) Int. Cl. G06Q 50/00; G06F 17/00; G06F 17/30; A61K 31/343

(52) U.S. Cl. 705/3; 705/2; 726/7; 715/205; 707/104.1; 707/E17.005; 707/E17.044; 514/469

(57) ABSTRACT

The present invention provides systems comprising two main components—a static and a dynamic component—that coupled with at least one interactive element of the systems permit providing information and support to a patient having a disease or disorder, responding to potential treatment issues of the patient, tracking a patient's treatment, and thereby improving patient compliance; as well as methods of using the systems, including methods of treating the patient and of ensuring the authenticity of a medication used by the patient.

### CROSS REFERENCE TO RELATED APPLICATIONS

Under 35 U.S.C. 119(e), this application claims the benefit of U.S. Provisional Application Nos. 60/971,975, filed Sep. 13, 2007, and 61/057,690, filed May 30, 2008, each of which are herein incorporated by reference in its entirety.

### FIELD OF THE INVENTION

The present invention relates to methods and systems for treating illnesses that typically require a patient to self-administer treatment and, in some instances, to a computer implemented method and system that dynamically tracks the patient's treatment and self-medication, and provides support to the patient.

### BACKGROUND OF THE INVENTION

In general, the failure of patients to comply with treatment in long-term clinical illnesses is a serious problem. Moreover, signs that the patient is getting better or worsening are sometimes missed or not identified in a timely and efficient fashion. For example, with respect to the illness of depression, non-compliance with a doctor-prescribed treatment program is a serious problem and more than half of all patients drop out of treatment within the first four weeks. There are many reasons for non-compliance, including but not limited to fears of drug dependencies, side-effects, or lack of efficacy. The side-effects can be difficult to understand from the patient's perspective; particularly, whether what the patient feels and experiences are typical of the illness, or if skipping or changing medication could improve the patient's situation with respect to the side-effects. To better help patients stick to treatment regimens, it is therefore important to address their specific concerns and problems that might lead to patient non-compliance.

Further, for example, Mind Yourself—The Lundbeck Mental Health Barometer 2008 report has revealed the most common symptoms amongst those with personal experience of depression are frequent thoughts of death or suicide (32%), low self esteem (29%) and sleep disturbance (28%). See e.g., “New Research Reveals 32% Of Those With Depression Experience Frequent Thoughts Of Death Or Suicide”, Medical News Today (May 13, 2008), available at www.medicalnewstoday.com/articles/107197.php (last accessed, May 13, 2008). The report has also revealed that when compared with other illnesses in terms of perceived disruptiveness, depression (59%) was rated above Parkinson's diseases (57%), heart disease (40%), anxiety disorder (29%), arthritis (24%), diabetes (16%) and asthma (14%). Id. Additionally, despite the fact that 88% of people think it is important to openly discuss depression, 72% do not think that it is easy to openly discuss and 60% think it would be a difficult condition to talk to a doctor about, 62% of people indicate that they would be embarrassed to discuss depression with their peer group and 77% of people believe that depression is not well understood. Id. Therefore, the depressive patient has a number of unmet needs, many of which can lead to unsuccessful treatment, patient non-compliance, or a combination of these or other treatment outcomes.

Furthermore, as more and more patients rely on prescribed medications for various illnesses, including depression, the sale of counterfeit medicines has become a serious and growing problem worldwide. Counterfeit sales not only cause lost sales by the original producers of the medicines but also put patients' lives at risk by circumventing the normal controls and procedures placed on the production and sale of such medicines. These types of sales can create dangerous situations, and ultimately undermine the confidence of the public in prescribed medications, and ultimately in their healthcare system. To efficiently help patients, it is therefore advantageous to provide a platform through which patients can gain access to the appropriate information and support, at the appropriate time, targeted to their individual situations, which also can determine the authenticity and validity of their medication. For example, by having the appropriate information (e.g., about drug dependency) at the appropriate time, the patient can more easily make a decision as whether to discharge this concern or proceed with further action. The present invention is directed to these and other important ends.

### SUMMARY OF THE INVENTION

The present invention relates to a computer implemented method of treatment of a disease or disorder in a patient in need thereof, wherein a computer system including an account database storing a plurality of patients' accounts is capable of communication via a network with the patient using at least one electronic device, the method comprises:

accessing a patient account for the patient, which is associated with login information, to retrieve patient data;

causing the display of a graphic user interface including at least a static information component including at least general information related to and based on a disease or disorder and a dynamic information component including at least an interactive element related to a stage of treatment of the patient determined from the retrieved patient data;

tracking patient treatment progress using at least patient information inputted in response to the interactive element of the dynamic information component;

dynamically generating a response based on the patient information; and

updating the patient data stored in the patient account based on the patient information.

The present invention also relates to a computer implemented method of treatment of a disease or disorder in a patient in need thereof, wherein as part of treatment of the patient, the patient has been prescribed to follow a regimen including taking a pharmaceutical composition, and compliance by the patient with the regimen is at least partially responsible for treatment success, the method comprises:

receiving a request including login information to access a system including an account database storing a plurality of patients' accounts, wherein the computer system is capable of communication via a network with the patient using at least one electronic device;

retrieving a patient account in response to receipt of the login information;

retrieving patient data from the patient account associated with the login information, the patient data including at least a record of the patient's prior interaction with the system; and

causing the display of a graphical user interface including at least a static information component based on the retrieved patient data, wherein the static information component includes at least general information related to the disease or disorder designed to increase patient compliance with the regimen of taking the pharmaceutical composition.

Further, the present invention relates to a computer implemented method of identifying a patient for the treatment of a disease or disorder, wherein the patient using at least one electronic device is communicatively connected to a computer system via a network, the computer system including an account database storing a plurality of patients' accounts and a identifier database storing a plurality of known identifiers, the method comprises:

receiving a request from the patient to create a patient account on the computer system, the request including login information including at least a unique identifier related to treatment of the patient;

comparing the unique identifier to the plurality of known identifiers;

granting access to the patient to create the patient account only after a positive match of the unique identifier and one of the plurality of known identifiers;

collecting information relevant to treatment of the patient; and

storing the information in the patient account.

The present invention also relates to a system for treating a disease or disorder, the system comprises:

a computer system including at least one computer and a related database, the computer system communicatively connected via a network to one or more patient computers and operative with a plurality of programming modules to:

retrieve a patient account in response to login information;

retrieve patient data from the patient account associated with the login information, the patient data including at least a record of the patient's prior interaction with the system;

cause the display of a graphic user interface including at least a static information component and a dynamic information component based on the retrieved patient data, wherein the static information component includes at least general information related to the disease or disorder and the dynamic information component includes at least an interactive element related to a stage of treatment of the patient determined from the retrieved patient data;

record one or more inputs transmitted by the patient in response to the dynamic information component;

generate a response to the one or more inputs; and

update the patient account to include a record of the one or more inputs.

The present invention further relates to a method of treating a disease or disorder in a patient in need thereof, the method comprising:

administering to the patient a pharmaceutical composition for use in the disease or disorder; and

providing the patient access to a computer system of the present invention.

Also, the present invention relates to a method improving patient compliance with a treatment regime for a disease or disorder, the method comprises providing a patient under the treatment regime with access to a computer system of the present invention, wherein the treatment regime includes self-administration of a pharmaceutical composition by the patient.

The foregoing summary is meant to be illustrative of various embodiments of the invention and is not meant to limit in any way either the inclusion of other embodiments disclosed herein or within the scope of the present invention. Various objects, features, aspects and advantages of the embodiments of the present invention will become more apparent from the following detailed description of the invention, along with the accompanying figures in which like numerals represent like components.

### BRIEF DESCRIPTION OF THE FIGURES

FIG. 1 is an overview of a system architecture in accordance with an embodiment of the present invention;

FIG. 2A is a first screenshot of a homepage in accordance with an embodiment of the present invention;

FIG. 2B is a second screenshot of a homepage in accordance with an embodiment of the present invention;

FIG. 2C is a third screenshot of a homepage in accordance with an embodiment of the present invention;

FIG. 3 is a questionnaire in accordance with an embodiment of the present invention;

FIG. 4 is a questionnaire in accordance with an embodiment of the present invention;

FIG. 5A is a chart of a patient's treatment progress in accordance with an embodiment of the present invention;

FIG. 5B is a chart of a patient's treatment progress in accordance with an embodiment of the present invention;

FIG. 5C is a graphical representation of a plurality of patients' answers to a questionnaire in accordance with an embodiment of the present invention;

FIG. 6 is a flow diagram of a task item in accordance with an embodiment of the present invention;

FIG. 7 is a timeline of a patient's interaction with an embodiment of the present invention;

FIG. 8 is a screenshot of a diary in accordance with an embodiment of the present invention;

FIG. 9A is a screenshot of a diary in accordance with an embodiment of the present invention;

FIG. 9B is a screenshot of a shared diaries in accordance with an embodiment of the present invention;

FIG. 10 is a screenshot of a webpage used to ask an expert a question in accordance with an embodiment of the present invention;

FIG. 11 is a screenshot of a webpage displaying various questions that were asked and answered in accordance with an embodiment of the present invention; and

FIG. 12 is a screenshot of a secure log-in webpage in accordance with an embodiment of the present invention.

FIG. 13 is tabular and graphical data comparing patient compliance at 16 weeks for patients of the present invention and overall patients.

FIG. 14 is other tabular and graphical data comparing patient compliance at 16 weeks for patients of the present invention and overall patients.

FIG. 15 is tabular data comparing patient compliance at 26 weeks for patients of the present invention and overall patients.

FIG. 16 is graphical data comparing patient compliance at 26 weeks for patients of the present invention and overall patients.

FIG. 17 is tabular data comparing patient compliance at 37 weeks for patients of the present invention and overall patients.

FIG. 18 is graphical data comparing patient compliance at 37 weeks for patients of the present invention and overall patients.

FIG. 19 is a graphical representation of the severity of depression of a group of patients of the present invention.

### DETAILED DESCRIPTION OF THE INVENTION

One aspect of the present invention provides a computer system, operative with programming and related databases, designed to provide a dynamic user interaction through the display of tailored information in response to one or more user inputs related to the user's disease or disorder.

An embodiment of the present invention includes, for example, a system for tracking a user's (e.g., a patient's) treatment program and causing the user to interact with the system, such that the system can identify and respond to potential treatment issues specific to the user. An embodiment of such system includes two main components—static and dynamic components—that when coupled with interactive elements of the system permit, e.g., the tracking of treatment of the user.

Another aspect of the present invention provides for a computer-implemented method of treatment of a disease or disorder in a patient in need thereof, the method comprising a system of the present invention. As will be further detailed herein, providing the interactive platform of the embodiments of the present invention increase the patient's likelihood of maintaining a treatment program, which includes taking medication for a doctor-prescribed period, and thus, improving patient compliance with treatment for the disease or disorder.

As used herein, unless otherwise indicated, the terms “medication”, “medicine” and “drug”, used alone or in combination with another term(s), are interchangeable and refer to a marketed pharmaceutical composition that is in a dosage form such as and without limitation, tablet, capsule, pill, lozenge, aerosol, dispersible powder, gel, granule, gum, implant, solution, suppository, suspension, trouche, or the like. As used herein, the phrase “pharmaceutical composition” refers to both marketed and unmarketed compositions comprising an pharmaceutically active ingredient and at least one pharmaceutically acceptable excipient without regard necessarily to form.

The term “treatment” or “treating” as used herein means curing, ameliorating or reversing the progress of a disease or disorder, or ameliorating or reversing one or more symptoms or side effects of such disease or disorder. “Treatment” or “treating”, as used herein, also means to inhibit or block, as in retard, arrest, restrain, impede or obstruct, the progress of a system, condition or state of a disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of a disease or disorder state, amelioration or palliation of a disease or disorder state, and remission of a disease or disorder, whether partial or total, detectable or undetectable.

A further aspect of the present invention provides for a method of treating a disease or disorder in a patient in need thereof, the method comprising administering to the patient a pharmaceutical composition for use in the disease or disorder; and providing the patient access to a computer system, wherein the computer system comprises a computer system of the present invention.

Another aspect of the present invention provides for a method of improving patient compliance with a treatment regime for a disease or disorder, the method comprising providing a patient under the treatment regime with access to a computer system, wherein the treatment regime includes self-administration of a pharmaceutical composition by the patient, and the computer system comprises a computer system of the present invention.

In certain embodiments, the invention provides a systematic approach to providing tools that enable a patient to get the right information at the right time.

In certain embodiments of the invention, a patient (i.e., a user) is invited to interact with a centralized computer system that is designed and configured to provide a number of dynamic elements and to keep a running timeline of the patient's treatment. An embodiment of the invention includes two main components: static and dynamic components. For example, when a patient signs up with the system, s/he enters her/his medication start date and is thereafter placed on a timeline. The timeline comprises, e.g., a “static” component and a “dynamic” component. The static component has general information about illness and medication and may include other elements related to the treatment of a disease or disorder. For instance, the static part is general information about depression, which has been divided into small sections which is sent over time to the patient at selected intervals during her/his treatment. For example, static messages may include, but not be limited to, inspirational tasks to motivate the patient to write in her/his diary, self-rating questionnaires, and medication queries. As used herein, the term “illness” refers to a disease, disorder, and the like. A dynamic timeline can be generated from input received by the system from the individual patient. Information is collected from the patient via the use of questionnaires (e.g., self-rating questionnaires and medication queries), although other methods as would be known to persons of skill in the art may be used. The system is programmed and designed to take particular actions in response to the information entered by the patient. In that way, the system ensures that the information in the patient's timeline will be targeted to that patient's unique situation.

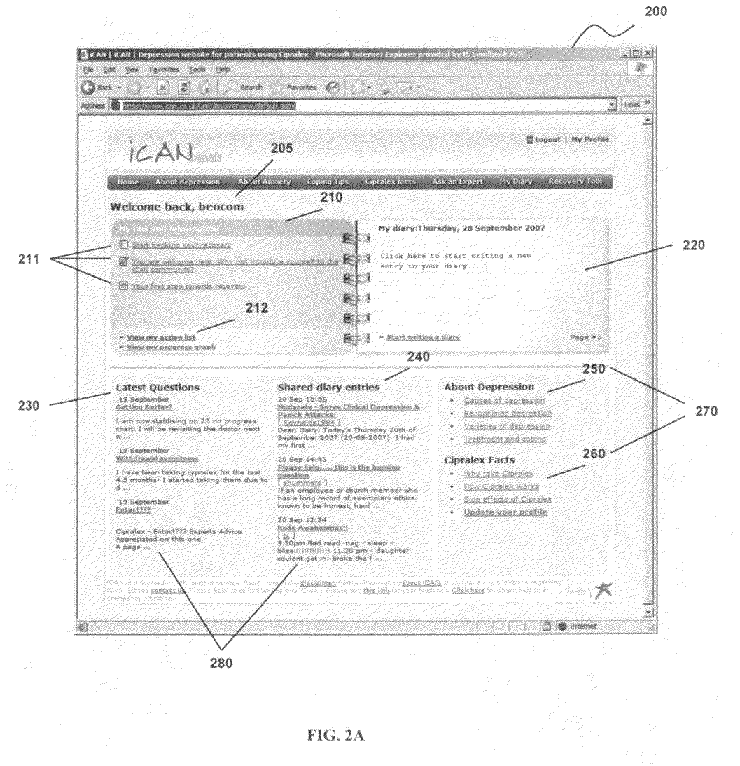

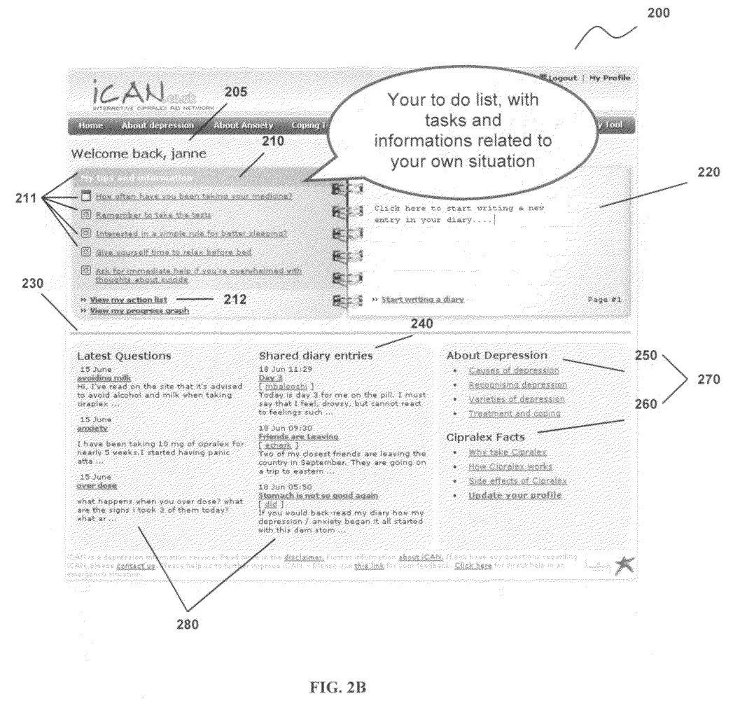

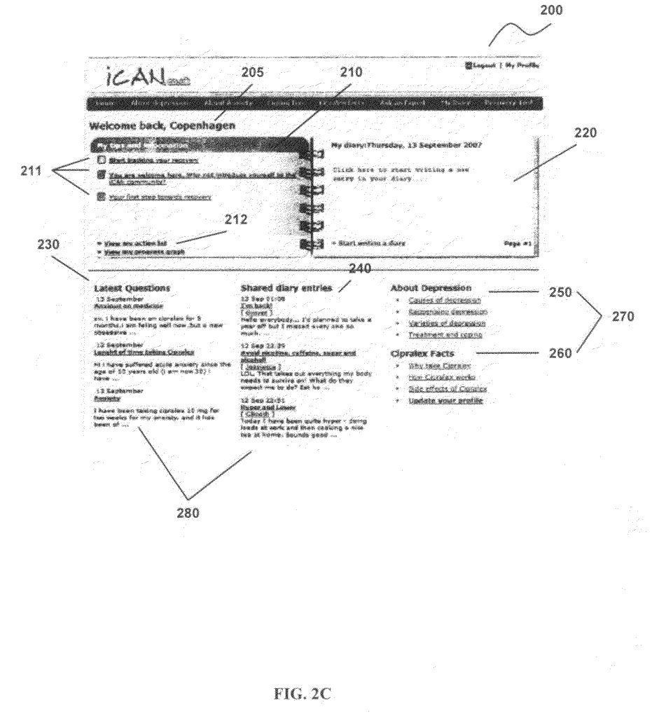

All of these components are designed to aid the patient in following the treatment prescribed by a doctor or other medical professional. FIGS. 2A-2C each depicts a home page of an embodiment of the system designed to provide multiple information and task windows for the patient. From this starting point, the patient can fulfill various tasks on her/his treatment timeline and/or interact with other patients, as described further below.

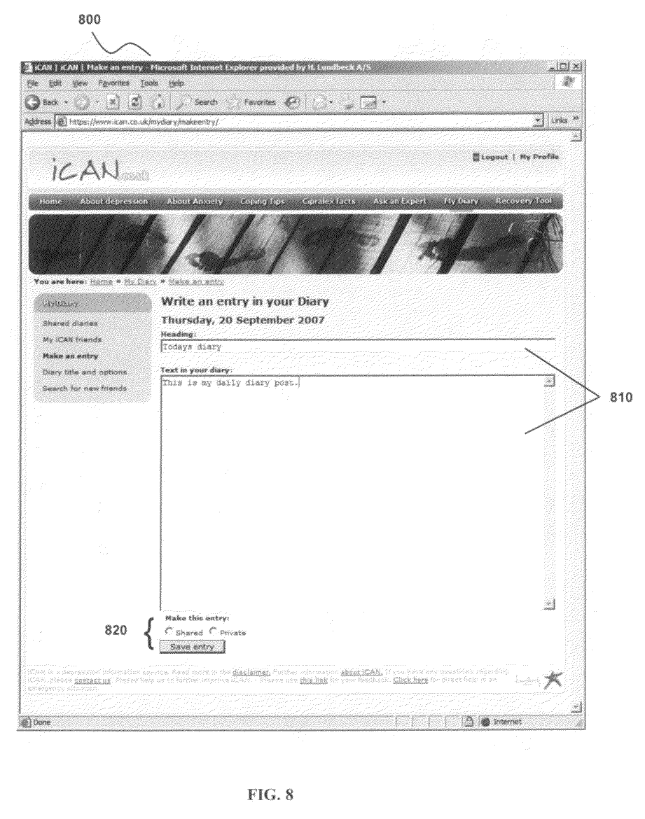

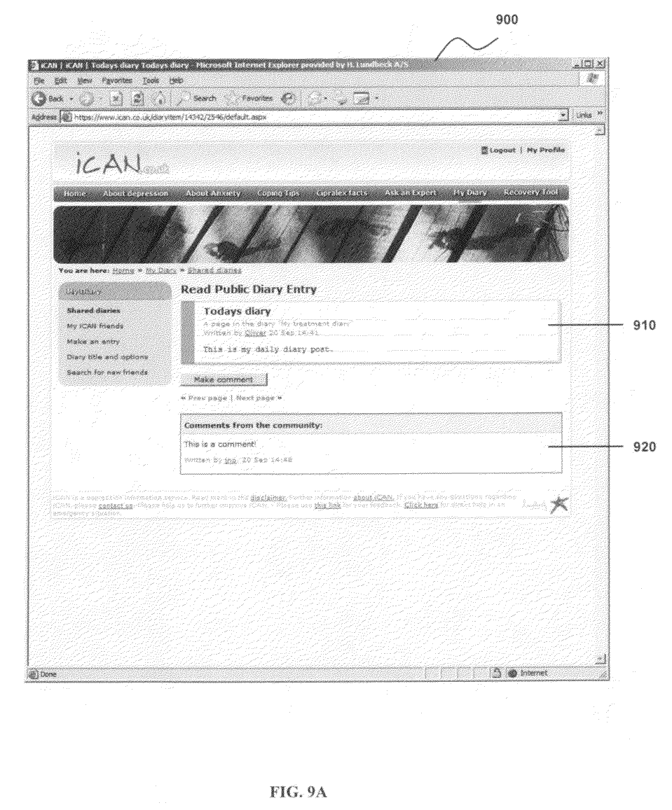

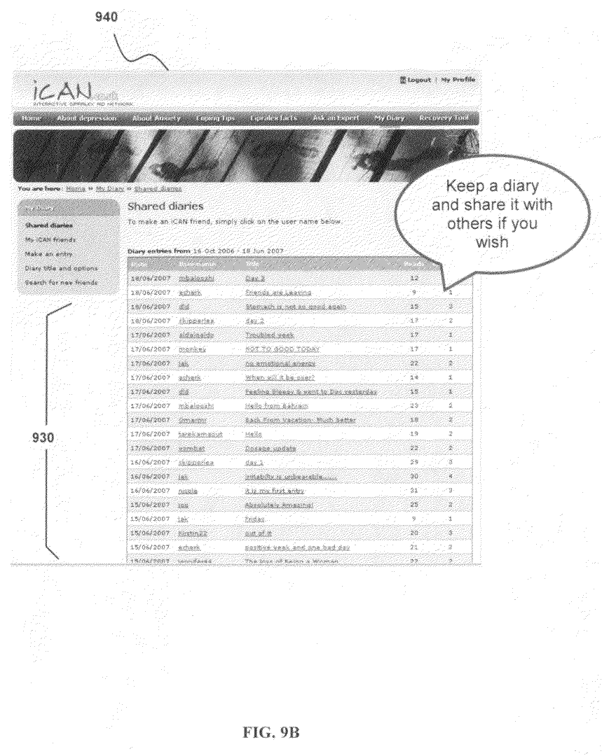

Certain embodiments of the present invention may also include a peer-to-peer function, such as a diary, which may be kept private or made public. In the system, there is an opportunity for patients to keep their own diary. In such embodiments, each patient has the ability to keep her/his diary private or to share it with others, e.g., share her/his public diary entries and have other patients comment on those entries. See, e.g., FIGS. 8, 9A and 9B. For example, when reading a “public” diary entry from another patient, patients have the possibility to comment on the entry and share their experience. Thus, when patients have concerns, worries and/or questions, they have a platform to write down in their diary and receive comments from other patients. The social networking and peer-to-peer function is considered to be a highly powerful motivator in keeping patients on their treatment path and reducing non-compliance, because advice from other patients who are or have been in the same situation are viewed as credible and highly valued responses by the asking-patient, who sometimes feels as others, including doctors, do not understand her/his predicament or how s/he is feeling.

Certain embodiments of the invention may also include a feature that keeps track of the patient's treatment progress by, for example, tracking the patient's depression levels, as determined from the patient's answers to the questionnaires.

Certain embodiments of the invention permit a patient to pose a question to an expert, such as a medical professional. In some such embodiments, the patient is permitted to review expert answers to questions posed by other patients.

In certain embodiments of the invention emergency information is provided to a patient, for example, when the patient is determined to be suicidal.

In certain embodiments of the invention, a computer implemented method includes, e.g., receiving account information transmitted by a user at a server of a computer system; using the account information to retrieve condition information related to the user that is stored in a database; causing the display of at least one action item at a user computer based on the condition information; receiving a user response to the at least one action item; retrieving a tailored system response from the database based on the user's response; and causing the tailored system response to be displayed at the user's computer. By way of non-limiting example, where the action item is a questionnaire, a user may be presented with the questionnaire upon login to the system, such that the user's state of illness, treatment or the like, can be tracked. In certain embodiments, the questionnaire includes a plurality of questions, thereby allowing that a tailored response can be displayed to the user to address specific user responses to the questionnaire. The details of illustrative questions and tailored responses are described further below. In certain embodiments, the action item can be related to information designed to monitor the user's medication usage, provide information related to the illness being treated or otherwise increase the user's use and enjoyment of the system.

In certain embodiments, the disease or disorder of the methods and systems of the present invention is a central nervous system disease or disorder, such as a cognitive or neurodegenerative disease or disorder. These diseases or disorders can be, but are not limited to, mood disorder, anxiety, schizophrenia (including schizoaffective disorders), Alzheimer's disease, Parkinson's disease, multiple sclerosis, Huntington's chorea, amyotrophic lateral sclerosis, Creutzfeld-Jakob disease, trauma-induced neurodegeneration, AIDS-induced encephalopathy, non-AIDS-induced infection-related encephalopathy, and a combination thereof. As used herein, the phrase “mood disorder” refers to any of several psychological disorders characterized by abnormalities of emotional state, such as, without limitation, bipolar disorders, depressive disorders, cyclothymic disorders, dysthymic disorders, mood disorders due to a general medical condition, mood disorders not otherwise specified and substance-induced mood disorders.

In certain embodiments, the cognitive or neurodegenerative disease or disorder is a mood disorder. In certain embodiments the mood disorder is depression (i.e., a depressive disorder). In certain embodiments, the depression can be, but is not limited to, atypical depression, bipolar depression, unipolar depression, major depression, endogenous depression (i.e., acute depression with no obvious cause), involutional depression (i.e., depression that occurs in mid-life or the elderly), reactive depression (i.e., depression caused by an obvious traumatic life episode), postpartum depression, primary depression (i.e., depression that has no obvious physical or psychological cause such as a medical illness or disorder), psychotic depression, and secondary depression (i.e., depression that seems to be caused by some other underlying condition such another medical illness or disorder).

In certain embodiments, the cognitive or neurodegenerative disease or disorder is an anxiety disease or disorder. In certain embodiments, the anxiety disease or disorder can be, but is not limited to, generalized anxiety disorder, panic anxiety, obsessive compulsive disorder, social phobia, performance anxiety, post-traumatic stress disorder, acute stress reaction, an adjustment disorder, a hypochondriacal disorder, separation anxiety disorder, agoraphobia, a specific phobia, anxiety disorder due to general medical condition, substance-induced anxiety disorder, and/or a combination thereof.

In certain embodiments, the central nervous system disease or disorder is a seizure disease or disorder that can be, but is not limited to, convulsion, epilepsy, status epilepticus, and/or a combination thereof.

In certain embodiments, the central nervous system disease or disorder is a neuropathic pain or migraine pain disease or disorder that can be, but is not limited to, allodynia, hyperalgesic pain, phantom pain, neuropathic pain related to diabetic neuropathy, neuropathic pain related to migraine, and/or a combination thereof.

In some embodiments, the central nervous system disease or disorder is a neuronal hyperexcitation state that can be, but is not limited to, neuronal hyperexcitation state in medicament withdrawal, a neuronal hyperexcitation state in intoxication, and/or a combination thereof.

Certain embodiments of the present invention will be discussed with reference to the aforementioned figures, wherein like referenced numerals will refer to like components. It should be noted that references in the specification to phrases such as “one embodiment” or “an embodiment” means that a particular feature, structure or characteristic described in connection with the embodiment is included in at least one embodiment of the invention. The phrases such as “in one embodiment” or “in certain embodiments” in various places in the specification are not necessarily, but can be, referring to the same embodiment. Although the methods and systems are referenced primarily in the context of depression and the medication sold under the trade name, CIPRALEX®, having the active ingredient escitalopram oxalate, it will be understood by persons of ordinary skill in the art that the present invention and its various embodiments could be applied to any clinical illness that requires a patient to self-medicate over a period of time. Moreover, it will be understood that the invention is not limited to any particular drug or drug type (i.e., dosage form).

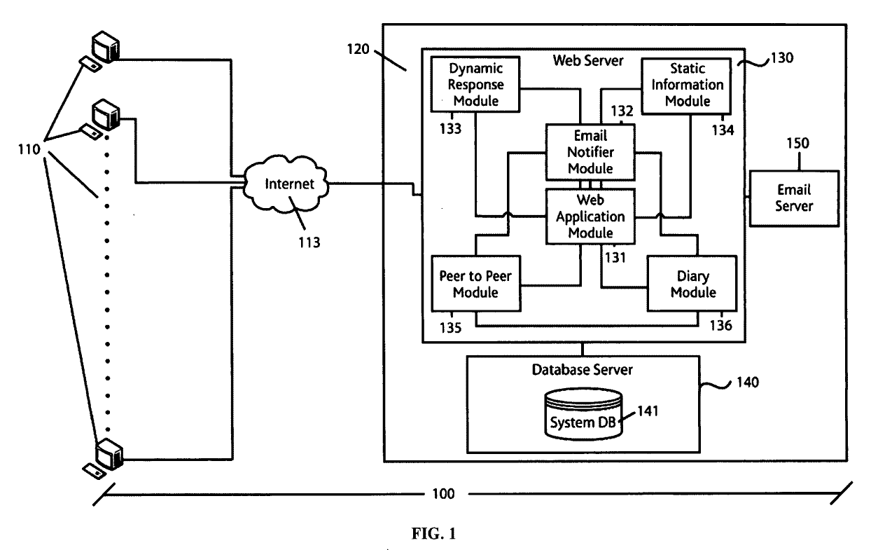

With reference to FIG. 1, there is shown and described an embodiment of a system 100 including computer systems, databases, and programming for providing functionality, as described herein, to one or more users. Server system 120, for example, includes one or more server and database systems in communication with one another. Server system 120 comprises at least a web server 130 and a database server 140 communicatively interconnected via an internal network, such as a local area network or LAN (not shown). Web server 130 includes programming, including various modules for receiving and retrieving information and generally providing interaction with a user (such as a patient), including but not necessarily limited to, a web application software module 131, an email notifier software module 132, a dynamic response software module 133 (i.e., a dynamic component), and a static information software module 134 (i.e., a static component). Server system 120 is also connected to an external network 113, such as the Internet, which enables server system 120 to communicate with one or more user computers 110. Database server 140 includes at least one system database 141. User computers 110 may gain access to the server system 120 via network 113.

It should be noted that although the embodiments described herein describe the use of one server and one related database for performing the various functions of server system 120, other embodiments could be implemented by storing the software or programming that operates the described functions on any combination of multiple servers and databases as a matter of design choice so long as the functionality described herein is performed. Although not depicted in the figures, web server 130 includes such art recognized components as are ordinarily found in server systems, including but not limited to processors, RAM, ROM, clocks, hardware drivers, associated storage, and the like. One skilled in the art will recognize, however, that because multiple users may be accessing such server at any given time it may become preferable to utilize multiple servers and databases, which may be used separately or in tandem to support the systems' traffic and processing, such as, by way of non-limiting example, a round-robin configuration utilizing multiple server systems. User computers 110 can be personal computers including at least a network adapter and web browsing capability, but may also include handheld devices such as PDAs, mobile smart phones (e.g., Blackberry®, iPhone®, Treo®, and the like), or other wireless devices.

It will also be understood that, although the various embodiments of the present invention described herein are being described in terms of web-based centralized server architecture, a thin client, fat-client, or peer-to-peer type arrangement could be substituted for the system architecture described herein and are within the scope of the present invention.

With reference again to FIG. 1, the functionality of the various programming modules of server system 120 will now be described. Web application software module 131 causes, e.g., generation of a plurality of graphic user interfaces (“GUIs”) (such as webpages), as shown for example in FIGS. 2-4 and 8-12. By way of example, information, received by server system 120 or generated by the dynamic response software module 133, peer to peer software module 135, static information software module 134, or diary software module 136, is processed to determine an appropriate response, as further described below. Based on such processing, web application software module 131 retrieves data stored in system database 141 or stored on the server system 120, and generates a GUI by incorporating the retrieved response information in a template interface. In other instances, a pre-designed GUI may be retrieved from system database 141 in response to a URL request received from a user. The pre-designed GUI is caused to be displayed by the web application software module 131 by transmitting the GUI data across network 113 to the user computer 110.

Dynamic response software module 133 is principally responsible for receiving and interpreting information transmitted to server system 120 by a user, and for determining an appropriate response thereto. Upon receipt of such information, dynamic response software module 133 accesses a table of potential responses to the received information, which, e.g., are stored in system database 141, and selects an appropriate response triggered by the received information in combination with additional user information that may have been previously collected and stored in system database 141. Dynamic response software module 133 then sends the appropriate response to web application software module 131. Using the response data, web application software module 131 can generate a webpage GUI using the response data, incorporating information received from dynamic response software module 133, information from system database 141, and any files (i.e., other information) that may be stored on web server 120.

Static information software module 134 principally functions to provide basic information about the illness being treated, the corresponding medication, and may include scheduled tips or action items (i.e., one or more task items) related to the illness. In operation, static information software module 134 accesses a table of static information stored in system database 141, and, when appropriate, it transfers such information to web application software module 131. Web application software module 131 can then generate a webpage GUI incorporating the static information received from dynamic response software module 133, information from system database 141, and any relevant files that may be stored on web server 120.

Diary software module 136 operates to provide a platform enabling users to make diary entries. Diary software module 136 further functions as to provide the GUI data and diary functionality to web application software module 131 so that users can keep a diary of their recovery progress. Diary software module 136 also allows users to view and edit their diary entries, and stores the edited diary entries in appropriate tables in system database 141.

Peer to peer software module 135 functions, e.g., to receive communications transmitted by users desiring to share information related to their treatment. For example, information may be entered by users, via their computers 110 and transmitted into a GUI (examples shown in FIGS. 8-11), generated by web application software module 131, across network 113. The communications (i.e., the information inputted by the users) are received by web server 120 and stored into appropriate tables in the system database 141. Peer to peer software module 135 also operates to provide the interface and functionality enabling users to input and review such information, and is further operative to permit access to such shared communications to any “allowed” user that requests such access. As such, peer to peer software module 135 controls and stores access information enabling a user to control who may access their shared information.

Email notifier module 132 enables the server system 120 to provide users with notifications via email. For example, a notification may inform the user that someone has commented on his/her diary. Such notifications may be automatically generated by email notifier module 132, or they may be based upon information sent to email notifier module 132 by the other modules of server system 120. After being generated, the notifications are transmitted to email server 150, which in turn transmits such notifications to one or more users as applicable. Email server 150 can be, e.g., a mail transfer agent (MTA), or system of MTAs, used to route email and act as a mail server, by storing email and supporting client access using, but not limited to, any of the following protocols: Post Office Protocol (POP), Internet Message Access Protocol (IMAP) or other protocols. Email server 150 responds to data and commands, sent by email notifier module 132, by sending emails to users. Email server 150 can be part of web server 130, or it can be separate. Users may retrieve the emails using a Mail User Agent (not shown), such as for example, Microsoft Outlook. In an embodiment, a mail user agent module (not shown) may be incorporated into server system 120.

It should be noted that although the embodiments described herein refer to multiple software modules for performing the various functions of system 100, other embodiments could be implemented with a single module incorporating the functions of several, or all, of the modules. The precise design of the software and the programming language used may be designed differently within the scope of the present invention. The software modules can be created using art recognized programming languages, including but not limited to ASP, Java, C#, ASP.NET, or PHP or any combination of known programming languages that allow the functionality described.

With reference now to FIG. 2A, there will be described the operation of system 100 as interacted with by a user of user computer 110 via network 113. FIG. 2A shows and describes an exemplary homepage 200, as generated by web application software module 131, that the user may confront upon accessing the server system 120 via the network 113. The homepage 200 includes, e.g., multiple sections or frames, which are populated by information generated by the various software modules of server system 120. For instance, homepage 200 can include a personal welcome message 205 and a personal tips and information element 210, which contains hyperlinks 211 that direct a user to various static or dynamic components, one of which may be, for example, an invitation for a user to fill out a questionnaire related to his/her depression. In the embodiment being described, the tips and information element 210 is generated by web application software module 131 based upon data sent to it by dynamic response software module 133. Homepage 200 may also include a shortcut to diary element 220, which enables a user to make diary entries into the diary GUI, the functionality of which is provided by diary software module 136. Homepage 200 may further include a static general information element 270, which provides all users with non-personalized hyperlinks 250 to information about their illness or non-personalized hyperlinks 260 to information about the medication they are taking. This information is sent by the Static Information module 134. Homepage 200 can further include a peer to peer communications shortcut element 280, which includes, e.g., snippets of peer to peer communications sent by the peer to peer software module 135, to the web application module 131.

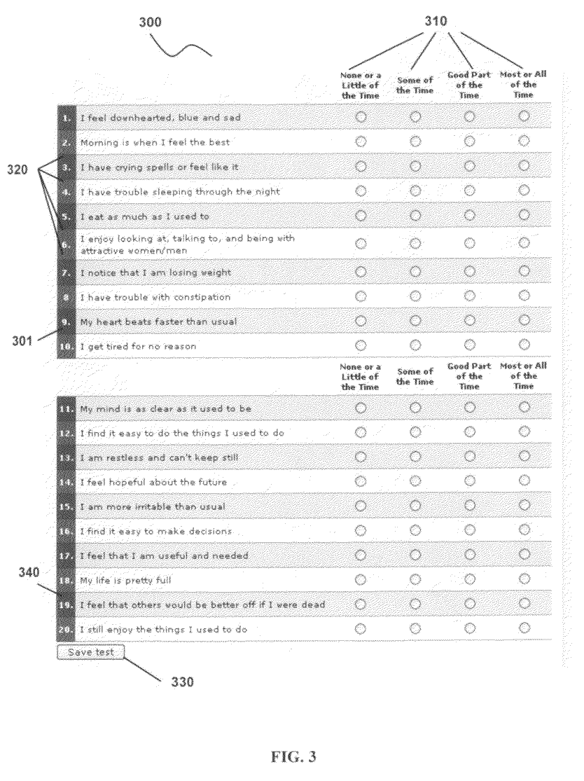

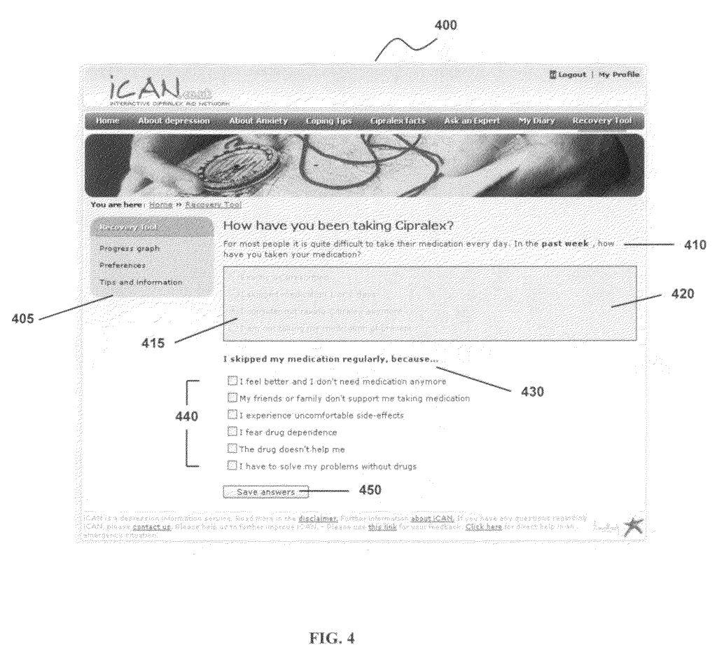

Personal tips and information element 210 of homepage 200 provides the user with various personalized assignment hyperlinks 211, the completion of the various personal assignments associated with these hyperlinks may assist in the user's recovery from depression, and assist the user's continued taking of the medication. These assignments are dynamically generated by dynamic response software module 133 based upon a combination of factors, including, but not limited to, the user's profile, the severity of the user's depression, and the length of time the user has been taking the medication or under treatment. For example, once the user has started the second week of treatment, dynamic response software module 133 of server system 120 will provide a user with an assignment to click a hyperlink and read a webpage, generated by web application module 131, describing how they should be feeling on the second week of the treatment. In another instance, dynamic response software module 133 of server system 120 may provide a user with a hyperlink to an assignment that is a task, such as that which can be seen in FIG. 3, the completion of which will assist in monitoring the user's depression by dynamic response software module 133; or the task seen in FIG. 4, which will assist in monitoring the user's progress on the medication or since under treatment by dynamic response software module 133.

Diary element 220 of homepage 200 has, e.g., the dual purpose of catharsis and advice, both of which may have a positive effect on a user's recovery, compliance with taking the medication and/or maintaining treatment. Users may feel better as a result of expressing their feelings by keeping an online diary. Additionally, server system 120 allows the users to make their diaries “public,” i.e., seen by other users. This allows the users to share their experiences, and also to get input from other users via “comments” that can be left by other users via a GUI, the functionality of which is provided, e.g., by peer to peer module 135 of the system. These comments can be a great help to a depressed individual, because others who have gone through similar experiences can provide support, insights and advice to the depressed individual. Overall, the various features provided by system 100 can have the effect of increasing a user's usage of system 100, and thus the user's usage of the appropriate medication and adherence to an appropriate treatment program. Additionally, “public” diary entries may be anonymous to permit users to speak freely and get valuable advice from fellow users. The diary can include an email service, the functionality of which is supplied by email notifier module 132, which may send the user an email if other users commented on their entries. There may also be further GUI containing webpages (not shown), functions of which are provided by diary software module 136, that have extended functionality such as, by way of non-limiting example, providing the ability to view previous diary entries, make one or more previous entries private, and erase and add comments. Furthermore, a compliance element (not shown) may be incorporated into diary software module 136, where diary software module 136 can instruct web application module 131 to display certain information tasks to the user, for example, an instruction to introduce herself/himself to the user community, or to write about her/his disease progress, or to write about problems s/he is experiencing.

An embodiment of peer to peer communications shortcut element 280 shows some of the latest questions 230 submitted by other users, and the latest shared diaries' entries 240. Users can click through each individual entry, or to a list of all of such entries in a separate peer communications elements (not shown).

FIGS. 2B and 2C further exemplify homepage 200 that a user may confront upon accessing server system 120 via network 113 as generated by web application software module 131.

In order to provide a user with useful personalized advice, information, and guidance, server system 120 requires the user to input information. With reference further to FIGS. 3 and 4, there is shown and described exemplary questionnaires 300 and 400 as interacted with by a user of user computer 110 via network 113. The results of the questionnaires are used for actions that include, but are not limited to, tracking the user's progress, providing advice, populating the personal messages and tips element 210 of the user's homepage, and providing the user with emergency contact information, e.g., if a high risk of suicide is detected.

For example, questionnaire 300 provides a list of questions that can be used to evaluate the user's level of depression. The self-rating questionnaire shown in FIG. 3 is based on the renowned Zung Self Rating Scale and gives the patient the opportunity to monitor his/her own illness over time. Using the Zung Self Rating Scale and the information entered by a patient over time, the system can dynamically respond to a worsening or improving situation.

In certain embodiments, web application module 131 provides the user with a GUI displaying the questions 320 and providing corresponding input fields 310 so that the user can answer questions 320. Input fields 310 may be, by way of non-limiting example, radio buttons, check boxes, text boxes, drop down boxes, or buttons. Users may submit the completed questionnaire 300 by, for example, clicking a button 330. The answers are then stored, e.g., in system database 141, and subsequently interpreted, according to a medically accepted methodology, such as a Zung Scale, by dynamic response software module 133. Dynamic response software module 133 then populates the personal messages and tips element 210 with the elements corresponding to the interpretation of the results of the questionnaire. In certain embodiments, the system will, e.g., give the patient an overall score related to the patient's level of depression based on his/her answers. In this self-rating-questionnaire there are some questions that are very important to detect and react to in the context of the patient's treatment or immediate well-being. For example, question 19 [340], which seeks information as to whether the patient believes that others would be better off if the patient was dead, can indicate a potential suicide. In this instance, if the patient answers, “Good Part of the Time” or “Most or All of the Time”, the system is designed, e.g., to react substantially immediately by sending an email with emergency contact information.

In certain embodiments, the system is able to react to dependencies within multiple answers from the patient. For example, if questions 1, 4 and 9, all have a negative score based on the patient's answers, the system is designed, e.g., to send out specific information addressing these issues. It either can be sent immediately or time+X amount of days. In certain embodiments, the system also is flexible so as to enable the inclusion of other self-rating questionnaires and to enable customized system responses to certain patient answers.

By way of non-limiting example, if the user is found to be depressed, the dynamic response software module 133 will place tasks into the personal messages and tips element 210 that may inform the user that depression at the early stages of treatment is normal, or to seek medical advice, depending on the severity of the depression.

Questionnaire 400 (a medication query), as shown in FIG. 4, is an example of another questionnaire a user may be confronted with. Questionnaire 400 shows an initial question 410 that the user answered by checking radio button input field 415 in the answer section 420. In the embodiment shown in FIG. 4, there are four (4) options for answering the question, “In the past week how have you been taking your medication?”:

1. Exactly as prescribed;

2. I skipped medication 1 or 2 days;

3. I consider not taking the medication anymore; or

4. I am not taking the medication at present.

If the patient answers #2, the system is designed, e.g., to provide information regarding the effect of skipping medication for a short period of time. This information may be placed on the patient's timeline, but is not necessarily provided to the patient at the time the medication query is provided.

However, if the patient answers #3 or #4, information will be, e.g., pushed to the patient as soon as such response is given. In the embodiment shown in FIG. 4, a window at the bottom of the query will open up to ask the patient why s/he has answered the questions in that fashion. This is where the system can receive specific reasons for potential non-compliance and react according to the patient's individual input.

In certain embodiments, the user's answer is stored in system database 141 and is interpreted by the dynamic response software module 133. As a result of the initial answer, the user may be confronted with a follow-up question 430, which asks the user to input information to clarify or expand on his/her answer to the initial question 410, via input fields 440. Questionnaire 400 may be used to find out information including, but not limited to, whether a user has been taking his/her medication, and if s/he has not, the dynamic response software module 133 can instruct the web application module 131 to generate a GUI to inform the user of the dangers of skipping doses and a subsequent proper course of action.

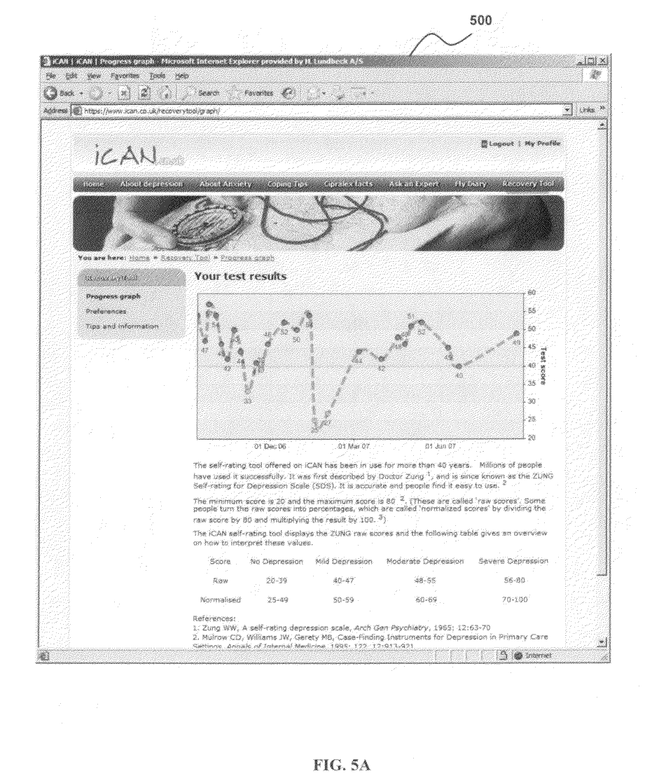

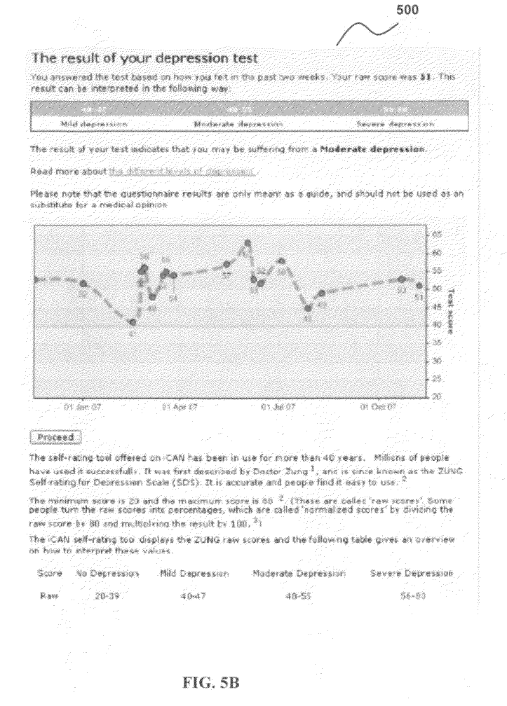

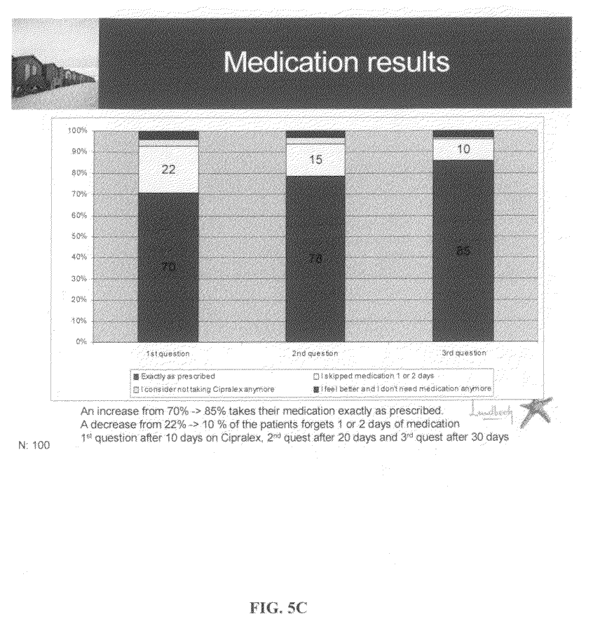

It may also be useful for a person suffering from a mental illness to see their progress mapped over time. As used herein, the phrases “mental illness” or “mental illnesses” refers to a group of diseases and disorders characterized by any one or any combination of the following: 1) a disease of the brain, with predominant behavioral symptoms, as in paresis or acute alcoholism; 2) a disease of the mind or personality, evidenced by abnormal behavior, as in hysteria or schizophrenia; 3) any psychiatric illness listed in Current Medical Information and Terminology of the American Medical Association; and 4) any illness listed in the Diagnostic and Statistical Manual (DSM-IV) for Mental Disorders of the American Psychiatric Association. For example, such a feature can allow the user to track his/her depression level over time and may be useful in identifying events that triggered an increased depressive state or helped the user overcome a low point. With reference further to FIGS. 5A and 5B, there is shown and described exemplary chart 500 of two user's treatment progress as interacted with by each user of user computer 110 via network 113. Server system 120 can, e.g., track the user's depression levels over time based upon the user's answers to various questionnaires (e.g., 300 and 400), which are stored in system database 141. Web application module 131 has, e.g., functionality to map this information over time to provide a user with a graphical representation or chart 500 of his/her treatment progress. It will be apparent to those skilled in the art, that although the chart is shown as being a line graph, it can also be a bar graph, a pie chart, or any suitable method of representing values over time.

Also, based on questionnaire 400, the system further can provide insight on general patient compliance behavior as shown by FIG. 5C, which graphically represents a plurality of patients' answers to questionnaire 400.

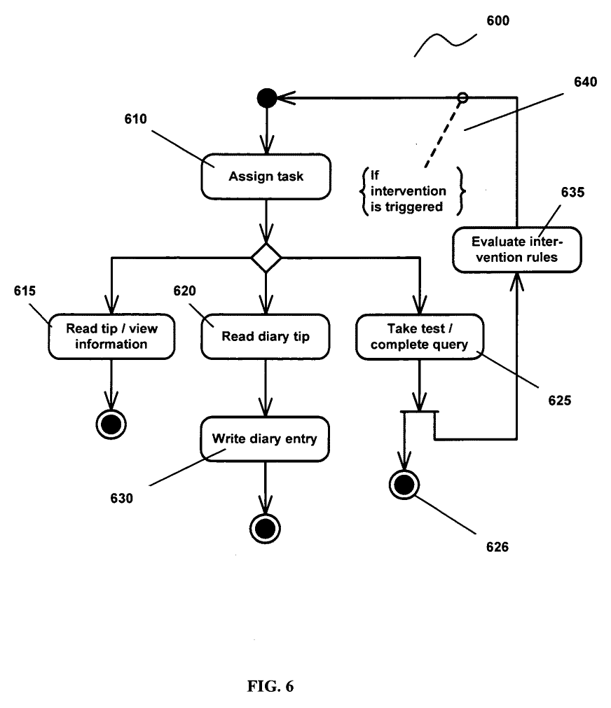

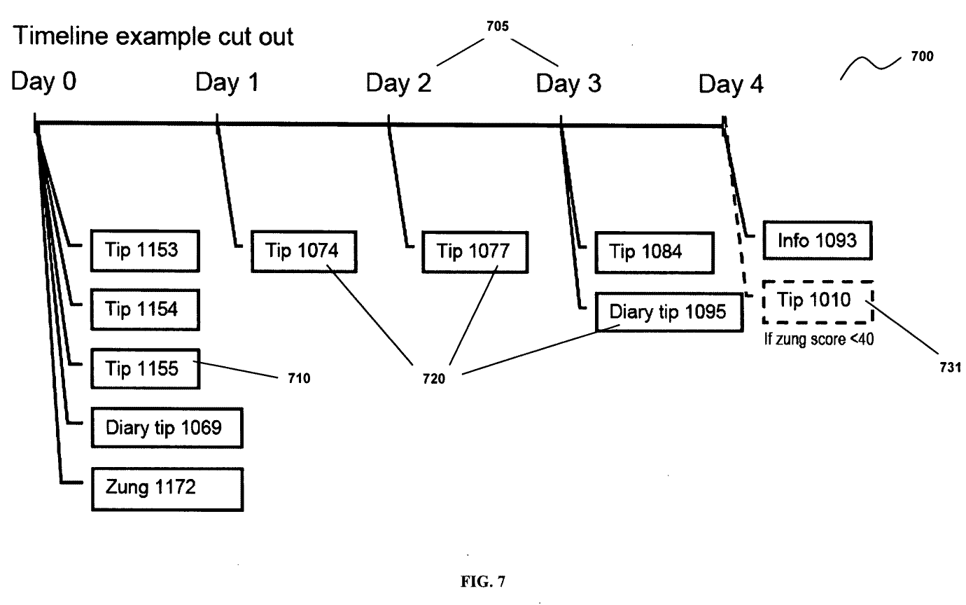

Dynamic response software module 133 runs, e.g., recursively where it repeatedly looks through the tables of system database 141 corresponding to a user who is logged into the system via the network 113 in order to determine whether there are any tasks to be assigned to such user. With further reference to FIG. 6, there is shown and described an embodiment of the process dynamic response software module 133 cycles through when it assigns tasks for the user to complete. Due to a triggering condition, such as a time event 705, as seen in FIG. 7, or a particular intervention event that may occur in step 640, dynamic response software module 133 assigns, e.g., a task to the user in assign task step 610. In step 610, dynamic response software module 133, chooses, e.g., an applicable task, and then cycles to the respective follow up step. These steps include, but are not limited to: read a tip and/or view information step 615, where a user is given an instruction to read a tip and/or view information; read a diary tip step 620, where the user is asked to read a tip related to the diary; or take a test and/or complete a query step 625, where a user is asked to take a test and/or complete a query. If the user was asked to read a diary tip in step 620, dynamic response software module 133 may then ask him/her to write a diary entry in step 630.

If the user is confronted with the take test and/or complete a query step 625, dynamic response software module 133 evaluates the results of the test and/or query in step 635, and issues an appropriate response by cycling to an appropriate follow up step. For instance, as a result of the evaluation performed in step 635, dynamic response software module 133 may cycle to issue intervention step 640, and immediately assign an intervention task 731. This may happen, for example, if a user completes a Zung self-rating questionnaire 300, and the program determines that the user may be suicidal. In such case, the dynamic response software module 133 will e.g., assign a task for the user to read information about being suicidal and provide the user with a telephone number to a suicide hotline. Persons of skill in the art will recognize that other types of responses, including notifying the user's doctor or appropriate authorities, may be configured within the scope of the present invention.

With further reference to FIG. 7, there is shown and described an example of a timeline 700 of interactions by a user of user computer 110 with server system 120 via network 113. Depending on the day of use 705, a user will be assigned a variety of tasks 710, such as reading tips or completing tasks, as provided by dynamic response software module 133. Depending on the day, different tips 720 may be shown. Additionally, tasks generated as result of user input 731 may be displayed. FIG. 7 presents an illustration based on assumed conditions, and various other timelines are possible depending on the disease, the user's profile, and other factors.

With further reference to FIGS. 8 and 9, there will be described and shown an example of the operation of diary module 136 as interacted with by a user of user computer 110 via network 113. When entering a diary entry the user is e.g., confronted with a diary entry page such as GUI 800 generated by web application module 131, functionality of which is provided by diary module 136. The user has to fill out a number of fields 810. By way of non-limiting example of the diary entry page GUI 800, the user has to complete the subject and text fields 810. After completing the diary entry, the user may choose to share the diary entry or to keep it private using, e.g., radio selection buttons 820. The diary entry is then transmitted to and stored in the appropriate tables of the system database 141. After completing the diary entry, the user may then view the entry 910 and, if the entry is made public, the user may view other users' comments about the entry 920, on the diary page GUI 900. As shown in FIG. 9B, a user also may view other patients' shared diary entries 930 on the shared diaries page GUI 940.

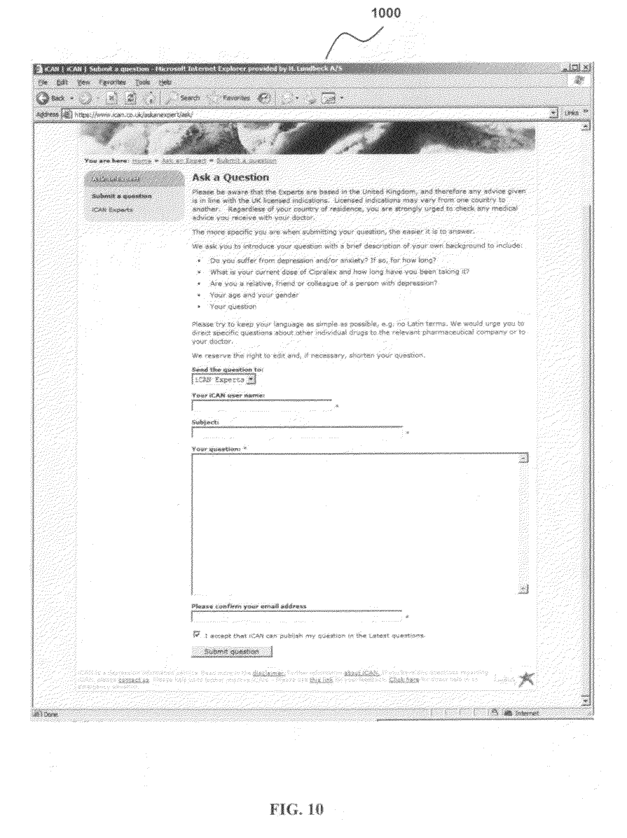

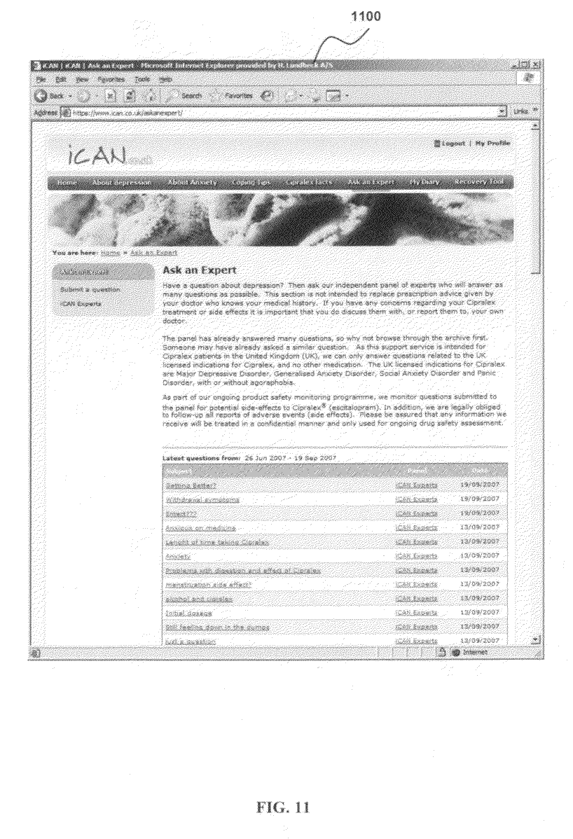

In order to give users the ability to obtain professional answers to their questions, peer to peer module 135 may provide users with ability to pose questions to an expert and to view other questions that experts have answered. With further reference to FIGS. 10 and 11, there will be described an example of the operation of an expert communication element, of the peer to peer module 135, as interacted with by a user of user computer 110 via network 113. The users of the system may, by filling out an expert question GUI 1000 generated by web application module 131, ask a question relating to their disease or medication. The question will thereafter be referred to and answered by an expert. Both the question and the answer are stored in the proper tables of system database 141. In order to prevent the same questions from being asked multiple times, both the questions and the answers may be accessible by users via archive GUI 1100 generated by the web application module 131. Other embodiments of the expert communication element may include live question and answer sessions with one or more experts (not shown).

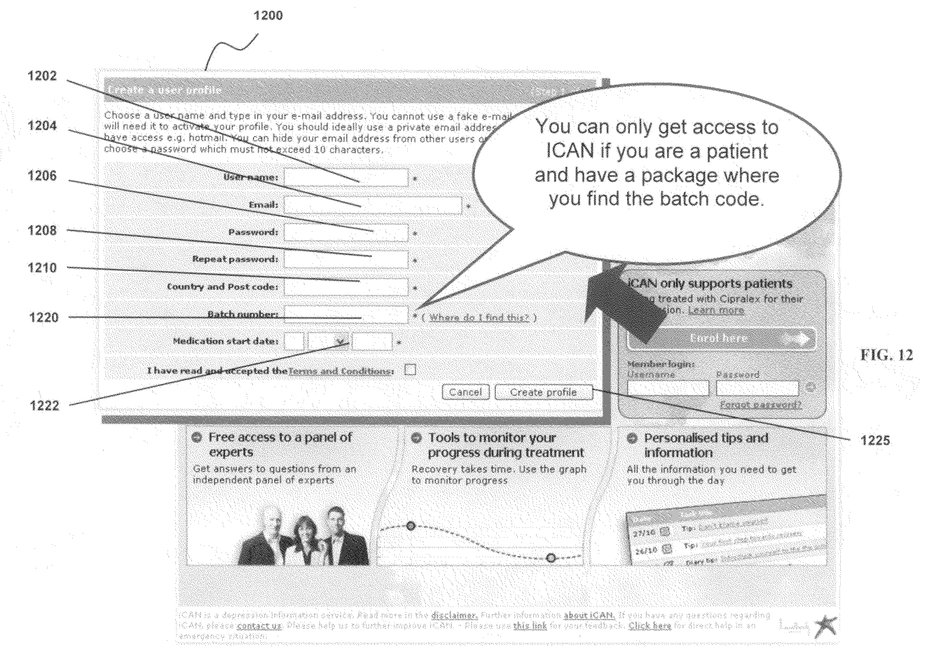

Because there are many regulations with respect to the communication of medical information to the general public on prescription medication, it is important to distinguish between patients and the general public. As such, an embodiment of the system ensures that the users are actual patients of the treatment for which the system is directed by requiring patients to enter a batch code from the pharmaceutical packaging in order to get access to the site. For example, with further reference to FIG. 12, there is shown and described an example of a security element of server system 120 as interacted with by a user of user computer 110 via network 113. Because depression and other mental illnesses are a very personal matter, the system may be open only to those on the particular medication for a particular disease or disorder. Therefore, before logging in, the users may be required to create an account by filling out a form 1200, with certain elements, such as the batch code from the medication, which can serve as a method of verification of actual illness. To increase security, form 1200, e.g., includes a number of fields designed to ensure the user is actually a person taking the required medication (i.e., the medication was prescribed for the user) and to prevent unauthorized access to the user's account. Thus, form 1200 includes, for example, but is not limited to, user name field 1202, email field 1204, password field 1206, repeat password field 1208, country code field 1210 (which can accept a country post code), batch code field 1220, and medication start date field 1222. Other profile information such as, and without limitation, MSN Messenger ID, homepage, gender, date of birth and short text about the user may also be collected. The user can also select precisely what information the user wants to make available to the rest of the community. The user creates her/his account by submitting such requisite information via create profile button 1225.

The batch code is a unique code found on the package labeling of the medication, such as, e.g., a batch number. Thus, as used herein, the phrases “batch code”, “batch number”, “serial number”, “medication identifier” are interchangeable. This information can be, e.g., verified by the web application module 131 against information in the system database 141, and then the user will be given a username and password, which will be stored in the system database 141. Thereafter the users will have to use their username and password to log into the system. It will be apparent to one skilled in the art that any number of security protocols may be used to keep the information and the system secure.

In another embodiment, the system 100 may be used to increase the security and validity of a pharmaceutical composition (i.e., a marketed drug product). In order to accomplish this, instead of, or in addition to, a batch number, medicines may be manufactured with an identifying serial number. Such a serial number may be configured to correlate to the medicine's packet size, dose, strength, manufacturing location, or any other relevant characteristic. The aforementioned data can be obvious from the configuration of the serial number, or, in the alternative, can be encrypted, along with other data, within the serial number. In such embodiment, for example, a user of the system 100, using a GUI, similar to the one described above with reference to FIG. 12, can input his/her medicine's serial number (i.e., the unique medication identifier). This information can be, e.g., verified by the web application module 131 against lists of valid serial numbers (i.e., a plurality of known medication identifiers), which can be stored within in the system database 141 or some other database specially configured for this purpose. If the serial number matches a valid serial number as correct (i.e., a positive match), the user will be informed by, for example, a GUI generated by web application module 131 that his/her medicine is valid. Alternatively, if the serial number on the medicine does not match a valid serial number (i.e., a non-match or a mismatch), the user will be informed by, for example, a GUI generated by web application module 131, that his/her medicine is invalid or that it may be counterfeit. In these cases, the user can be provided with further instructions, such as, for example, to call his/her doctor or pharmacy, or the pharmaceutical company that manufactures the drug. Additionally, if an invalid serial number is detected, the system 100, e.g., stores the invalid number in a table of invalid numbers, in the system database 141 or other specially designed database.

In such embodiment, the system 100 also can be configured to match the serial number of the medicine to the user's individual account to determine whether the medicine corresponds to the user's prescribed regimen—for example, whether each dose is the proper strength, whether the medicine is the one prescribed, or whether any other relevant property is correct or incorrect. Additionally, submissions of serial numbers to the system 100 may be performed in any number of ways in which electronic information can be transmitted through a network including, but not limited to, via telephone, email, GUI, photo submission, scanning, and others similar methods.

Since counterfeiters may attempt to copy legitimate serial numbers, in certain embodiments, the system 100 may keep track of how many times a specific number has been entered, and if it is entered more then once, the system will, e.g., place the serial number into the invalid serial number table in the system database 141, so that this number may not be used in the future.

In further embodiments, system 100 can be configured to detect various information from the serial numbers inputted by users in order to analyze such data. By way of non-limiting example, if medicine is manufactured with serial numbers that correlate to certain geographical regions, system 100 can analyze where most of the medicine used is purchased from. Additionally, if serial number correlates to specific dosages, the system 100 may analyze and determine the most popular (e.g., most frequently prescribed) dosage(s). Furthermore, in certain embodiments, system 100 may collect other information from users, via GUI generated by web application module 131, and link that information to information collected from the serial numbers. For example, system 100 may access the user's home address or request that the user input the location (including retail location) where he or she purchased medicine. This information can be used to identify geographical regions, or retail chains, where sales of counterfeit or invalid medicine may have occurred, and may be used by pharmaceutical companies, alone or in combination with law enforcement, to take action against the sale of counterfeit products. For instance, in one example, the affected pharmaceutical company can run an advertising campaign in the affected geographical location. As used herein, the terms, “counterfeit”, “counterfeited”, “invalid” and the like, includes products that are “gray goods” and “parallel imports”, where gray goods is the phrase used in the U.S. and parallel imports the phrase used in the European Union to designate legitimately marketed products that are unofficially acquired abroad and then imported into another country against the wishes of the product owner.

To ensure that counterfeiters cannot circumvent the verification process by printing the same serial number(s) as on the original package(s), in certain embodiments, the system can be, e.g., configured to verify the same serial number as “correct” only for a predetermined number of tries. The level of tolerance may be adjusted, for example, based on the use and type of drug, such as a highly narcotic drug may have a tolerance of “1,” whereas a less dangerous drug may have a tolerance of “two” or more. Thus, if a drug has a tolerance of “2,” and is submitted more than two times, the user can be informed that the serial number has been used before and that s/he can be advised to contact the owner pharmaceutical company for further information and advice.

It should be noted that the analyses of the information collected described herein are exemplary and any other type of data-mining or analysis may be performed on the information. It further should be noted that the serial numbers may be any type of identifying number in any form or means, for example, they may be typed numbers, RFID chips or tags, bar-codes, and any other method of encoding information.

### EXAMPLES

It has been found that the use of the system 100 is highly effective in keeping patients on their medication regimen. It has been shown that people using an embodiment of the system of the invention are more likely to stay on medication, and also consume more of their medication. Conservative analyses of user data to a medication query (questionnaire 400 depicted in FIG. 4) were made as follows.

### Data Preparation

1. Data Used for the Analysis

The data generated by users filling in a medication questionnaire, a self-rating questionnaire and a registration form were used in order to analyse the compliance of users of the system of the invention. The raw data set included all observations until (and including) 28 Aug. 2007 (see Analysis 1, below); an extended analysis included all observations generated until (and including) 17 Jun. 2008 (see Analysis 2, below).

2. Program Start Date

When registering for an account with a system of the invention, new users are asked to declare when they started taking medication (i.e., CIPRALEX®). Because users who inputted a medication start date that lied in the future, i.e., if the user had not started to take CIPRALEX®, are not subsequently queried by the system as to whether they started taking CIPRALEX® when they receive subsequent medication queries, which is exactly like all other users, the medication start date for these users was adjusted to the date when they filled out a medication query declaring being on CIPRALEX® for the first time.

3. Medication Start Earlier than Four Weeks Before Registration

Patients who had been taking antidepressants for more than four (4) weeks before registering for an account with a system of the invention were dropped from the data set and the compliance of new CIPRALEX® users was analyzed. Some users reported up to 10 years of antidepressant use upon registration.

### Outcome Measure

The medication query asked users if they have been taking their medication regularly. There were four possible answers:
1. Exactly as prescribed;
2. I skipped medication 1 or 2 days;
3. I consider not taking CIPRALEX® anymore; or
4. I am not taking my medication at present.

Throughout the analysis, it was assumed that answers (1) and (2) signal compliance, whereas answers (3) and (4) signal non-compliance. It is of course possible that a user reporting to not take their medication at a certain point in time fills in another medication query later and reports taking it as prescribed. It was not assumed that patients having been non-compliant once have completely stopped further treatment.

A fact of the system was that while users are reminded to fill in a medication query every ten (10) days, they are free not do so; and thus, the interval lying between two answered medication queries can be longer than 10 to 14 days. Taking the answers as inputted would imply assuming that the user had been compliant over the whole period, when however it may be that the user had not returned to the system because s/he had not been compliant and therefore did not use system or did not want to admit this in a medication query, and consequently did not answer the intervening medication queries. But, if these users were simply dropped from the analysis, this would result in a highly selective data set with likely an unbelievably high compliance rate.

To avoid bias in the analysis caused by this fact, and in order to get a very conservative estimate, it was chosen to not drop these users but to keep them in the sample and—under certain circumstances—to not take their answers as given. This meant that if there lied more than a pre-specified amount of time in between two filled in medication queries, an answer indicating compliance is re-set to non-compliance. The maximum accepted time interval lying between two filled in medication queries was set to be 14 days. If, e.g., a user reported taking their medication on August 5^{th}, and gave the same answer on August 20^{th}, then it was still assumed that s/he had been non-compliant between those two dates. If this user fills in the medication queries within intervals of 14 days or less in the future and reports to be compliant, these future observations are used as they are.

This results in a quite conservative evaluation of patient compliance since it also could be the case that a user who in reality has been compliant is considered to have been non-compliant due to the number of days between two filled in medication queries exceeding 14. In other words, the compliance rate that is calculated may underestimate compliance, but it never overestimates compliance, given the assumption that people truthfully complete the medication query.

### Estimation of Compliance Curve

Using this data on compliance, user compliance rate for each treatment week was calculated up to 16 weeks of treatment. The relationship between treatment length and compliance rate was estimated using the natural logarithm of the number of treatment weeks as explanatory variable in a simple linear regression.

### Comparison to IMS Data

The calculated compliance curve was compared to the compliance curve from a joint analysis of H. Lundbeck A/S (Valby, D K) and IMS Health Incorporated (Corporate Headquarters, Norwalk, Conn., USA; EMEA Regional Headquarters, London, UK) regarding compliance, using data from the IMS Prescriptions Insights database (herein also referred to as, “IMS Study”). Herein, the phrase “overall patient(s)” refers to the average individual prescribed CIPRALEX® for the period from 1 Mar. 2004 to 28 Feb. 2005 and represented by IMS Prescriptions Insights data used in the joint analysis. Thus, as used herein, “overall patient(s)”, “patient(s) in general”, “patient(s) of the IMS study”, “average IMS patient”, and the like, are interchangeable.

### Robustness Checks

Adjusting the program start date did not substantially affect the results of the analysis.
Excluding patients that have been on an antidepressant for more than four weeks when registering with the system of the invention did not substantially affect the results of the analysis.
Using a logarithmic function instead of a linear or quadratic function did not substantially affect the results of the analysis.

### Users of the Invention

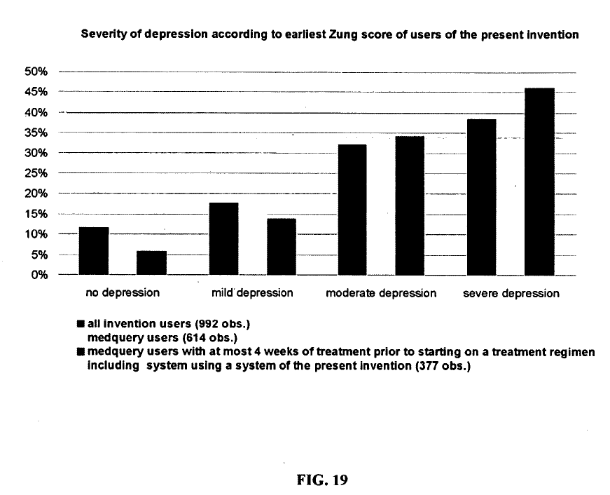

More than two-thirds of all users of a system of the invention, also referred to herein as “patients of the invention”, had a moderate or severe depression according to their Zung score based on when they filled in for the first time the Zung self-rating questionnaire. See e.g., FIG. 19.

Analysis 1: Up to about 4 Months (16 Weeks)

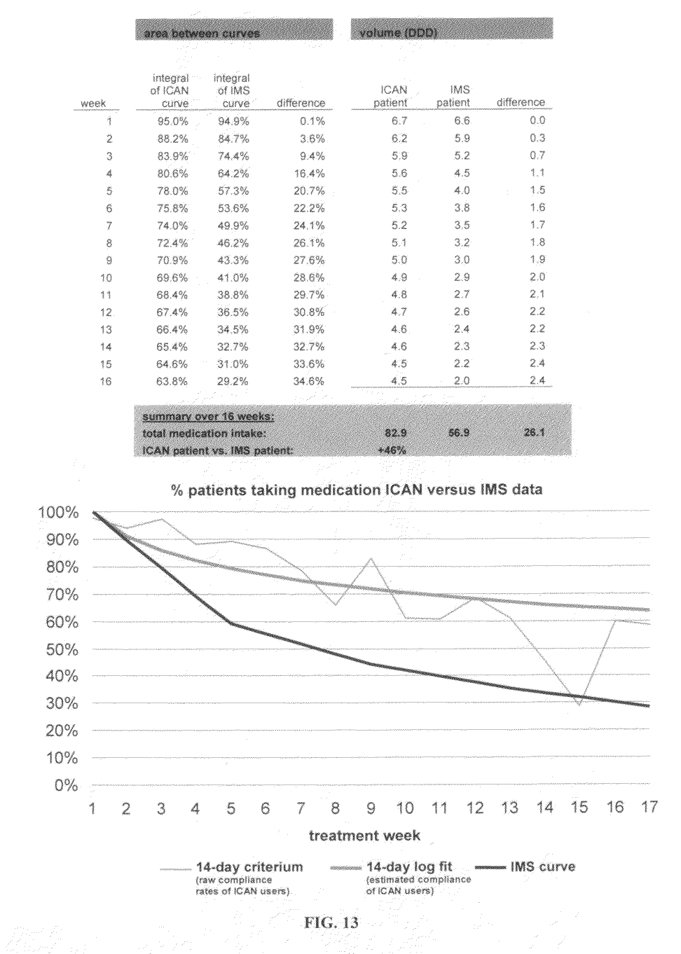

The following refers to the analysis that is based on the data generated until (and including) 28 Aug. 2007. As shown in FIG. 13, over the first 16 weeks of treatment, a fully compliant patient would need 16*7=112 DDDs (Defined Daily Dosages). Compared to this, the patients in the analysis based on IMS data use on average about 57 DDDs, whereas a patient of the invention on average uses about 83 DDDs. The difference of about 26 DDDs means that a patient using the system of the invention consumed about 46% more CIPRALEX® than an overall patient (average IMS patient), and those about 26 DDD represent about 46% of what patients in the IMS study consume within the same period.

As can be seen in FIG. 13, in the 5^{th} week of treatment, patients using the system were found to be about 21 percentage points more likely to be taking their medication than patients in the IMS study, and this increased to about 35 percentage points in week 16. Over the first 16 weeks of treatment this sums up to the 46% higher medication intake reported above.

Analysis 2: Up to about 9 Months (37 Weeks)

The following refers to the analysis that is based on the data generated until (and including) 17 Jun. 2008. The analysis was performed using the data points for the first 16 weeks (in order to replicate the first analysis), the first 26 weeks and the first 37 weeks of treatment, respectively.

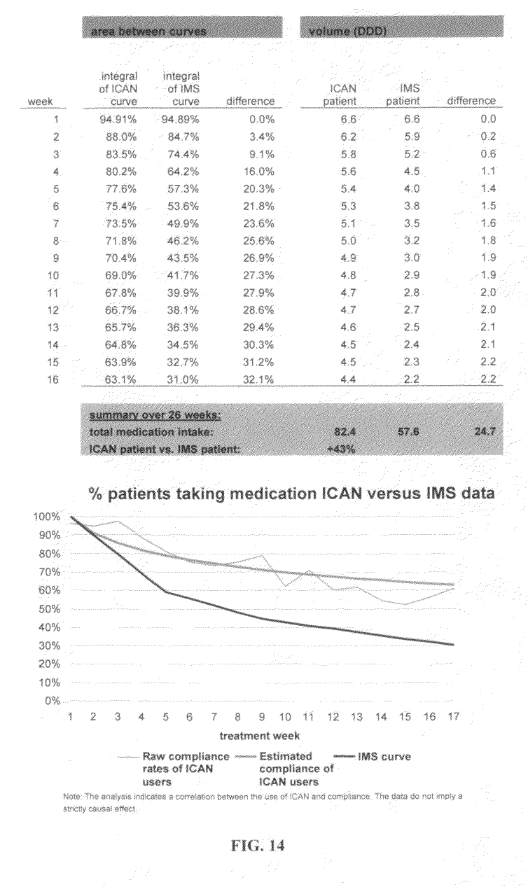

Within the first 16 weeks of treatment, an average patient of the invention consumed about 25 DDD more than an overall patient (average IMS study patient), which equates to about 43% more CIPRALEX® than the overall patient, and those about 25 DDD represent about 43% of what patients in the IMS study consume within the same period. See FIG. 14. This result shows that the results of the previous analysis are not substantially affected by using a larger dataset, which includes more observations that are collected over a wider period of time.

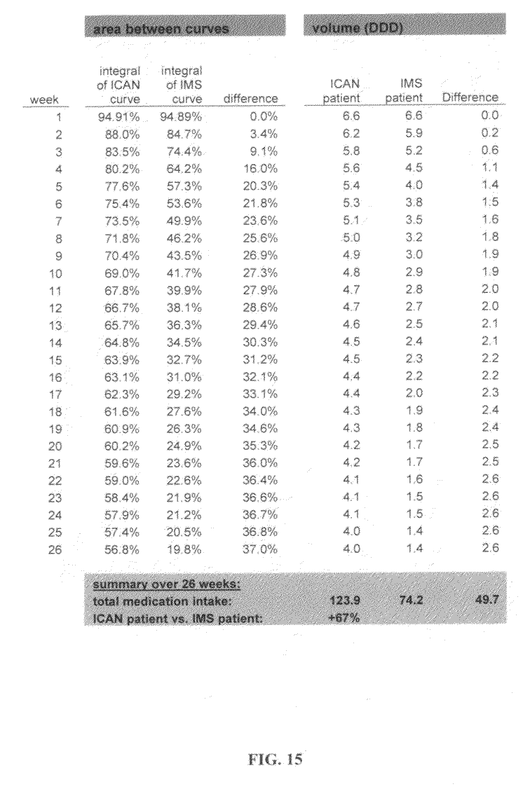

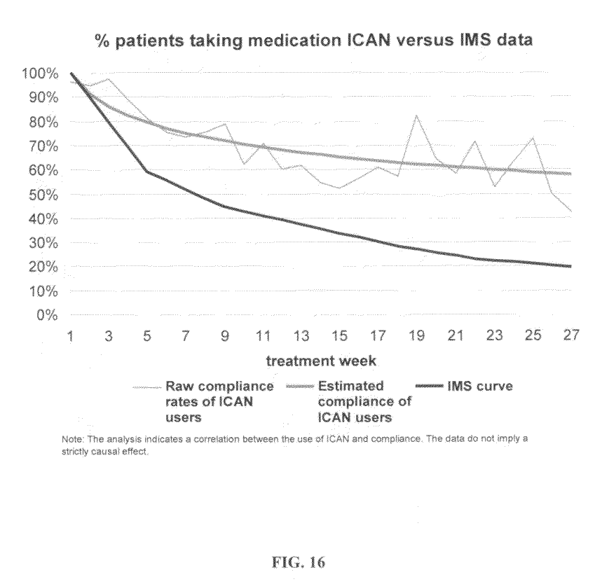

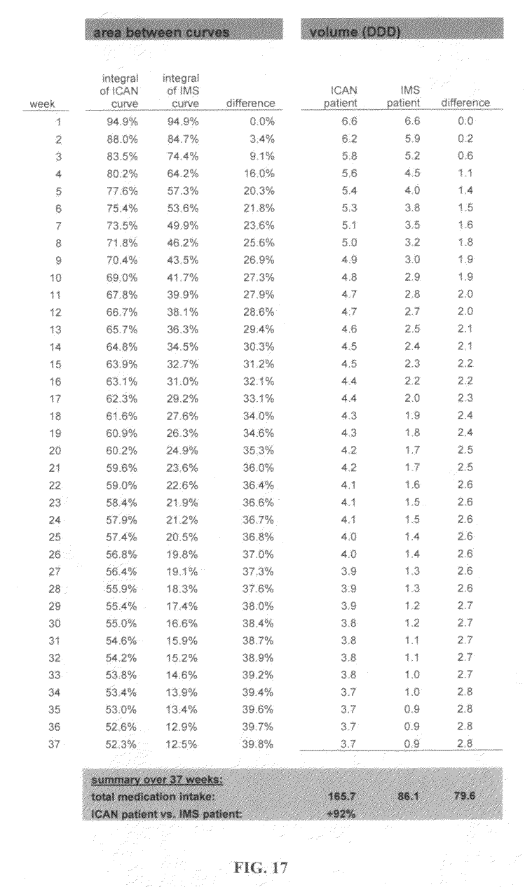

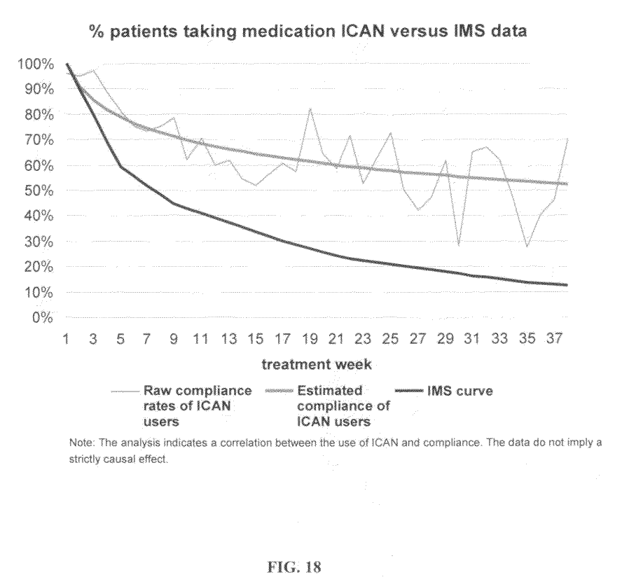

Further, as shown in FIGS. 15 and 16, within about the first six (6) months of treatment, an average patient using the system consumed about 50 DDD more than an overall patient (average IMS study patient), which equates to about 67% more CIPRALEX® than that consumed by the overall patient, and those about 50 DDD represent about 67% of what patients in the IMS study consume within the same period. Within about the first nine (9) months of treatment, an average patient using the system consumed about 80 DDD more than an overall patient (average IMS study patient), which equates to about 92% more CIPRALEX® than that consumed by the overall patient, and those about 80 DDD represent about 92% of what patients in the IMS study consume within the same period. See FIGS. 17 and 18.

As shown in FIGS. 14-18, as the average patient of the invention continues being treated with a CIPRALEX® regimen including a system of the invention, the patient continues to be more compliant than the average patient in general (i.e., than the average patient of the IMS study). Additionally, the difference in compliance between the average overall patient and the average patient being treated with a CIPRALEX® regimen including a system of the invention increases over time.

It will be appreciated by those skilled in the art that the systems of the present invention may incorporate other software modules, such as instant messaging between users, or other software modules that may assist users with staying with their treatment. The need for these software modules may be determined by, e.g., the type of illness or treatment. For example, a treatment that requires group therapy may incorporate a chat room software module where users can congregate and post dynamically at once.

It will be further appreciated by those skilled in the art that the figures are purely illustrative, and that the system may be implemented in any number of ways, by the actual designers, as long as the functionality, relating to the treatment of a disease or disorder, such as mental illness, through a web based system, remains the same. Moreover, while there have been shown and described fundamental novel features of the invention as applied to the embodiments thereof, it will be understood that omissions and substitutions and changes in the form and details of the disclosed invention may be made by those skilled in the art without departing from the spirit of the invention.

Each reference cited in the present application, including literature references, books, patents and patent applications, is incorporated herein by reference in its entirety.

What is claimed is:

1. A computer implemented method of treatment of a disease or disorder in a patient in need thereof, wherein a computer system including an account database storing a plurality of patients' accounts is capable of communication via a network with the patient using at least one electronic device, the method comprising:
accessing a patient account for the patient, which is associated with login information, to retrieve patient data;
causing the display of a graphic user interface including at least a static information component including at least general information related to and based on the disease or disorder and a dynamic information component including at least an interactive element related to a stage of treatment of the patient determined from the retrieved patient data;
tracking patient treatment progress using at least patient information inputted in response to the interactive element of the dynamic information component;
dynamically generating a response based on the patient information; and
updating the patient data stored in the patient account based on the patient information.

2. The method of claim 1, wherein at least one of the static information component and the dynamic information component is designed to improve patient compliance with a treatment program.

3. The method of claim 2, wherein the treatment program includes a regimen for taking a pharmaceutical composition.

4. The method of claim 1, wherein the disease or disorder is a central nervous system disease or disorder.

5. The method of claim 4, wherein the central nervous system disease or disorder is a mood disorder.

6. The method of claim 1, wherein the computer system further includes a medication identifier database storing a plurality of known medication identifiers for a pharmaceutical composition, and the method further comprises:
receiving login information from the patient including at least a unique medication identifier; and
granting the patient access to the patient account only after a positive match of the unique medication identifier and one of the known medication identifiers in the medication identifier database.

7. The method of claim 6, wherein the unique medication identifier is printed on packaging containing at least one dosage form of the pharmaceutical composition or on a surface of an individual dosage form of the pharmaceutical composition.

8. The method of claim 6, further comprising providing at least one medication reminder to the patient based on the unique medication identifier.

9. The method of claim 1, wherein the tracking step further comprises transmitting at least a portion of the patient information to a medical professional.

10. The method of claim 9, further comprising receiving treatment information from the medical professional and providing the treatment information to the patient.

11. The method of claim 1, further comprising displaying one or more task items related to the stage of treatment of the patient determined from the retrieved patient data.

12. The method of claim 11, wherein at least one task item is associated with a hyperlink, and the method further comprises:
receiving an indication from the patient to retrieve a webpage designated by the hyperlink; and
causing the webpage to be displayed to the patient, the webpage containing an element related to the task item to be performed by the patient.

13. The method of claim 12, wherein the element provides static information relating to the disease or disorder.

14. The method of claim 13, wherein the element is designed to improve patient compliance with a treatment program.

15. The method of claim 14, wherein patient compliance with the treatment program is improved by about 40% to about 50%.

16. The method of claim 11, wherein:
the system further comprises a data tracking component; and
the tracking step further comprises collecting data from a plurality of patients relating to the plurality of patients' compliance with each of the plurality of patients' treatment programs.

17. A computer implemented method of treatment of a disease or disorder in a patient in need thereof, wherein as part of treatment of the patient, the patient has been prescribed to follow a regimen including taking a pharmaceutical composition, and compliance by the patient with the regimen is at least partially responsible for treatment success, the method comprising:
receiving a request including login information to access a system including an account database storing a plurality of patients' accounts, wherein the computer system is capable of communication via a network with the patient using at least one electronic device;
retrieving a patient account in response to receipt of the login information;
retrieving patient data from the patient account associated with the login information, the patient data including at least a record of the patient's prior interaction with the system; and
causing the display of a graphical user interface including at least a static information component based on the retrieved patient data, wherein the static information component includes at least general information related to the disease or disorder designed to increase patient compliance with the regimen of taking the pharmaceutical composition.

18. The method of claim 17, wherein:
the regimen further includes a schedule according to which the patient is directed to take the pharmaceutical composition; and
the static information is a reminder to take the pharmaceutical composition.

19. The method of claim 18, wherein the reminder further comprises a task item including a selectable element for tracking completion of the task item.

20. The method of claim 19, wherein:
the system further comprises a data tracking component; and
the method further comprises collecting from a plurality of patients data related to the plurality of patients' compliance with each of the plurality of patients' treatment programs based at least in part on each of the patients' completion of the task item.

21. The method of claim 17, further comprising:
displaying a dynamic information component, wherein the dynamic information component includes at least an interactive element related to a stage of the patient's treatment determined from the retrieved patient data;
recording one or more inputs transmitted by the patient in response to the dynamic information component;
generating a response to the inputs;
updating the patient account to include a record of the inputs; and
tracking the patient's usage of the system.

22. The method of claim 21, wherein at least one of the static information component and the dynamic information component are designed to improve patient compliance with a treatment program.

23. The method of claim 22, wherein patient compliance is improved by about 40% to about 50%.

24. A computer implemented method of identifying a patient for the treatment of a disease or disorder, wherein the patient using at least one electronic device is communicatively connected to a computer system via a network, the computer system including an account database storing a plurality of patients' accounts and a medication identifier database storing a plurality of known medication identifiers, the method comprising:
receiving a request from the patient to create a patient account on the computer system, the request including login information including at least a unique identifier related to treatment of the patient;
comparing the unique identifier to the plurality of known identifiers;
granting access to the patient to create the patient account only after a positive match of the unique identifier and one of the plurality of known identifiers;
collecting information relevant to treatment of the patient; and
storing the information in the patient account.

25. The method of claim 24, wherein the unique identifier is printed on packaging containing at least one dosage form of the pharmaceutical composition or on a surface of an individual dosage form of the pharmaceutical composition.

26. The method of claim 24, further comprising:
receiving the unique identifier from the patient; and
collecting location information about the patient.

27. The method of claim 26, wherein the location information is derived from the patient's IP address or at least one entry of information by the patient.

28. The method of claim 26, wherein:
the computer system further comprises a trend tracking database; and
the method further comprises storing the unique identifier and the location information about the patient in the trend tracking database.

29. The method of claim 28, wherein:
the computer system further comprises a data tracking component; and
the method further comprises analyzing the location information in the data tracking component to identify at least one trend.

30. The method of claim 29, wherein the at least one trend identifies at least one location having:
a unique identifier that fails to match any of the plurality of known identifiers; or
a concentration of unique identifiers that fail to match any of the plurality of known identifiers.

31. The method of claim 30, wherein the at least one trend identifies at least one location having a concentration of gray market goods or counterfeit goods.

32. A system for treating a disease or disorder comprising:
a computer system including at least one computer and a related database, the computer system communicatively connected via a network to one or more patient computers and operative with a plurality of programming modules to:
retrieve a patient account in response to login information;
retrieve patient data from the patient account associated with the login information, the patient data including at least a record of the patient's prior interaction with the system;
cause the display of a graphic user interface including at least a static information component and a dynamic information component based on the retrieved patient data, wherein the static information component includes at least general information related to the disease or disorder and the dynamic information component includes at least an interactive element related to a stage of t treatment of the patient determined from the retrieved patient data;
record one or more inputs transmitted by the patient in response to the dynamic information component;
generate a response to the one or more inputs; and
update the patient account to include a record of the one or more inputs.

33. The system of claim 32, wherein at least one of the static information component and the dynamic information component are designed to improve patient compliance with a treatment program.

34. The system of claim 32, further comprising a medication identifier database storing a plurality of known medication identifiers, and wherein the plurality of programming modules are further operative to:
receive login information from the patient including at least a unique medication identifier; and
grant access to the patient account only after a positive match of the unique medication identifier and one of the plurality of known medication identifiers in the medication identifier database.

35. The system of claim 34, wherein the plurality of programming modules are further operative to:
track patient treatment progress using at least one patient information input in response to the interactive element of the dynamic information component;
receive a diary entry from the patient; or
display one or more task items related to a stage of treatment of the patient determined from the retrieved patient data.

36. The system of claim 35, wherein at least one task item is associated with a hyperlink; and the plurality of programming modules are further operative to:
receive an indication from the patient to retrieve a webpage designated by the hyperlink;
cause the webpage to be displayed to the patient, the webpage containing an element related to the task item; and
display the task item so as to indicate accomplished upon completion by the patient of the element.

37. The system of claim 36, wherein the element is designed to improve patient compliance with a treatment program.

38. The system of claim 37, wherein patient compliance is improved by about 40% to about 50%.

39. A computer implemented method of treatment of a disease or disorder in a patient in need thereof, wherein two or more patients use an electronic device capable of communicatively connecting to a computer system via a network and the computer system includes an interactive communication component, the method comprising:
providing a prompt to a first patient to communicate information related to the disease or disorder of the first patient;
receiving at least one communication related to the disease or disorder from the first patient;
displaying the at least one communication such that it is viewable by a second patient with access to the computer system;
receiving a responsive communication from the second patient in response to the communication from the first patient; and
displaying the responsive communication to the first patient.

40. The method of claim 39, further comprising monitoring the communications by a medical professional.

41. The method of claim 40, further comprising providing medical information by the medical professional to the first patient based on information in the communication.

42. A method of treating a disease or disorder in a patient in need thereof, the method comprising:
administering to the patient a pharmaceutical composition for use in treating the disease or disorder; and
providing the patient access to a computer system;
wherein the computer system includes at least one computer and a related database, the computer system communicatively connected via a network to one or more patient computers and operative with a plurality of programming modules to:
retrieve a patient account in response to login information,
retrieve patient related data from the patient account associated with the login information, the patient related data including at least a record of the patient's prior interaction with the system,
cause the display of a graphic user interface including at least a static information component and a dynamic information component based on the retrieved patient related data, wherein the static information component includes at least general information related to the disease or disorder and the dynamic information component includes at least an interactive element related to a stage of treatment of the patient determined from the retrieved patient related data,
record one or more inputs transmitted by the patient in response to the dynamic information component,
generate a response to the inputs, and
update the patient account to include a record of the inputs.

43. The method of claim 42, wherein the disease or disorder is a mood disorder.

44. The method of claim 43, wherein the pharmaceutical composition comprises escitalopram oxalate.

45. A method of improving patient compliance with a treatment regime for a disease or disorder, the method comprising:
providing a patient under the treatment regime with access to a computer system, wherein:
the treatment regime includes self-administration of a pharmaceutical composition by the patient, and
the computer system includes at least one computer and a related database, the computer system communicatively connected via a network to one or more patient computers and operative with a plurality of programming modules to:
retrieve a patient account in response to login information,
retrieve patient related data from the patient account associated with the login information, the patient related data including at least a record of the patient's prior interaction with the system,
cause the display of a graphic user interface including at least a static information component and a dynamic information component based on the retrieved patient related data, wherein the static information component includes at least general information related to the disease or disorder and the dynamic information component includes at least an interactive element related to a stage of treatment of the patient determined from the retrieved patient related data,
record one or more inputs transmitted by the patient in response to the dynamic information component,
generate a response to the inputs, and
update the patient account to include a record of the inputs.
